(19) United States
(12) Patent Application Publication
(10) Pub. No.: US 2022/0061785 A1
(43) Pub. Date: Mar. 3, 2022

(54) COMFORTABLE BARRIER CLIP FOR INTRA-ORAL DIGITAL X-RAY SENSOR

(71) Applicant: Keesha McCloud, Douglasville, GA (US)

(72) Inventor: Keesha McCloud, Douglasville, GA (US)

(21) Appl. No.: 17/412,251

(22) Filed: Aug. 25, 2021

Related U.S. Application Data
(60) Provisional application No. 63/070,148, filed on Aug. 25, 2020.

(51) Int. Cl. A61B 6/14; A61B 6/00

(52) U.S. Cl. CPC A61B 6/145; A61B 6/563

(57) ABSTRACT

Provided is a comfortable, intra-oral digital x-ray sensor device suitable for receiving a digital x-ray sensor therein. Also provided is a kit comprising the x-ray sensor device and a digital x-ray sensor.

### CROSS-REFERENCE TO RELATED APPLICATIONS

This application claims priority to U.S. Provisional Application No. 63/070,148, filed Aug. 25, 2020, which is incorporated by reference in its entirety.

### BACKGROUND

X-ray technology has been used by dental professionals to examine teeth and other dental structures for some time. Historically, a dental professional would strategically place a slide of x-ray film contained within a sheath into a patient's mouth and expose the film to an x-ray source. This method of dental x-ray was not well tolerated by patients because the film and sheath were uncomfortable. The film and sheath were not designed to contour to the patient's mouth and teeth.

Today, most dental offices have replaced traditional x-ray film technology with digital x-ray sensors. Like the older x-ray films and sheaths, however, modern digital x-ray sensors are uncomfortable, often featuring jagged square edges and hard surfaces that can protrude into a patient's soft palate. Accordingly, a need exists for a device capable of reducing the discomfort caused by intra-oral digital x-ray sensors. These needs and others are met by the following disclosure.

### SUMMARY

In one aspect, this disclosure relates to a dental x-ray sensor device comprising lingual and labial inner and outer side walls and a biting surface, the lingual and labial inner side walls and the biting surface together defining a channel configured to receive one or more teeth of a person; and a sensor bed between the labial inner and outer side walls configured to hold a digital x-ray sensor.

In a further aspect, this disclosure relates to a kit comprising (a) a dental x-ray sensor device comprising lingual and labial inner and outer side walls and a biting surface, the lingual and labial inner side walls and the biting surface together defining a channel configured to receive one or more teeth of a person; and a sensor bed between the labial inner and outer side walls configured to hold a digital x-ray sensor; and (b) a digital x-ray sensor.

Still other objects and advantages of the present disclosure will become readily apparent by those skilled in the art from the following detailed description, which is shown and described by reference to preferred aspects, simply by way of illustration of the best mode. As will be realized, the disclosure is capable of other and different aspects, and its several details are capable of modifications in various respects, without departing from the disclosure. Accordingly, the description is to be regarded as illustrative in nature and not as restrictive.

### BRIEF DESCRIPTION OF THE DRAWINGS

The accompanying drawings, which are incorporated in and constitute a part of this specification and together with the description, serve to explain the principles of the disclosure.

FIG. 1A is a perspective view of the labial side of an exemplary embodiment of the dental x-ray sensor device.

FIG. 1B is a perspective view of the lingual side of the dental x-ray sensor device.

FIG. 2A depicts exemplary embodiments of a generally rectangular x-ray sensor device.

FIG. 2B depicts exemplary embodiments of a generally circular x-ray sensor device.

FIG. 3 depicts an exemplary view of the labial side of the dental x-ray sensor device comprising a digital x-ray sensor in the sensor bed of the device.

### DETAILED DESCRIPTION

The present invention can be understood more readily by reference to the following detailed description of the invention and the Examples included therein.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present devices, methods, and kits may be understood more readily by reference to the following detailed description of preferred embodiments and the examples included therein.

While aspects of this disclosure can be described and claimed in a particular statutory class, such as the system statutory class, this is for convenience only and one of skill in the art will understand that each aspect of this disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or description that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### A. Definitions

Listed below are definitions of various terms used to describe this invention. These definitions apply to the terms as they are used throughout this specification, unless otherwise limited in specific instances, either individually or as part of a larger group.

As used in the specification and in the claims, the term “comprising” can include the aspects “consisting of” and “consisting essentially of.”

As used in the specification and claims, the singular forms “a,” “an,” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

“Optional” or “optionally” means that the subsequently described event or circumstance may or may not occur, and that the description includes instances where said event or circumstance occurs and instances where it does not.

As used herein, the term “substantially,” in, for example, the context “substantially free of” refers to a composition having less than about 10% by weight, e.g., less than about 5%, less than about 1%, less than about 0.5%, less than about 0.1%, less than about 0.05%, or less than about 0.01% by weight of the stated material, based on the total weight of the composition.

It is further understood that the term “substantially,” when used in reference to a composition, refers to at least about 60% by weight, e.g., at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature, component, or a combination of the components. It is further understood that if the composition comprises more than one component, the two or more components can be present in any ratio predetermined by one of ordinary skill in the art.

“Lingual,” as used herein, refers to the surface of a patient's tooth that is nearest the tongue. Thus, a “lingual side wall” refers to a side wall that is nearest the patient's tongue when the disclosed device is inserted into the patient's mouth.

“Labial,” as used herein, refers to the surface of a patient's tooth that is nearest the lips. Thus, a “labial side wall” refers to a side wall that is nearest the patient's lips when the disclosed device is inserted into the patient's mouth.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

### B. Intra-Oral Barrier Clip for Digital X-Ray Sensor

In one aspect, disclosed is a comfortable, flexible barrier clip designed to house an intra-oral digital x-ray sensor device. The barrier clip can be inserted into a dental patient's mouth and provide a barrier between the uncomfortable digital x-ray sensor and the structures of the patient's mouth. In one aspect, the flexible barrier clip is a dental x-ray sensor device comprising lingual and labial inner and outer side walls and a biting surface. The lingual and labial inner side walls and the biting surface can together define a channel configured to receive one or more teeth of a person. The device can also include a sensor bed between the labial inner and outer side walls. The sensor bed can be configured to hold a digital x-ray sensor.

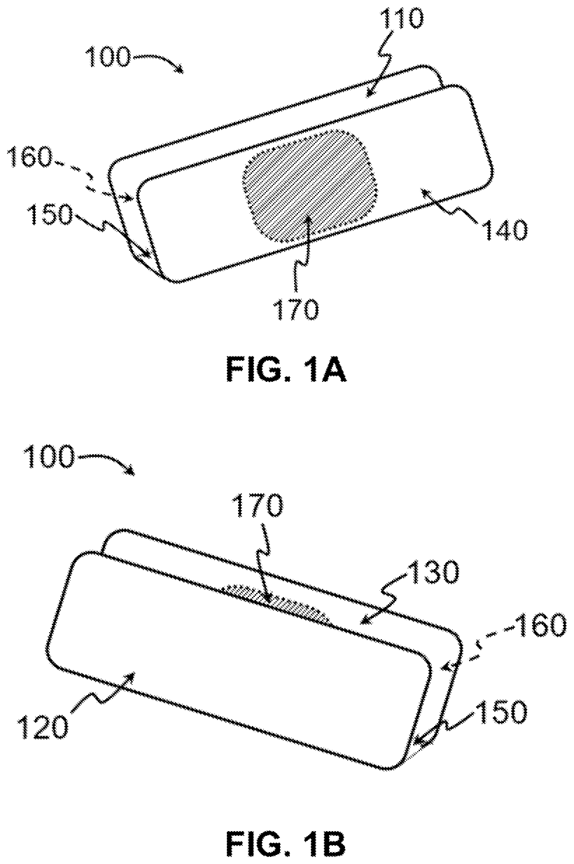

With reference to FIGS. 1A-B, an exemplary digital x-ray sensor device 100 can comprise a lingual inner side wall 110, as depicted in FIG. 1A, and a lingual outer side wall 120, as depicted in FIG. 1B. When digital x-ray sensor device 100 is inserted into a patient's mouth, the lingual inner and outer side walls 110 and 120 will be nearest the patient's tongue. Digital x-ray sensor device 100 also comprises a labial inner side wall 130, as depicted in FIG. 1B, and a labial outer side wall 140, as depicted in FIG. 1A. When device 100 is inserted into a patient's mouth, the labial inner and outer side walls 130 and 140 will be nearest the patient's lips.

Device 100 also includes a biting surface 150, which can be configured to engage one or more teeth, e.g., molar teeth, of the patient. The lingual inner side wall 110 and the labial inner side wall 130, together with biting surface 150, can together define channel 160. Channel 160 can be configured to receive one or more teeth of the patient when device 100 is inserted into the patient's mouth.

Device 100 further comprises sensor bed 170. Sensor bed 170 can be configured to hold a digital x-ray sensor. Thus, sensor bed 170 can comprise an opening and be configured as a pouch suitable for receiving a variety of shaped digital x-ray sensors. For example, sensor bed 170 can be configured to hold a rectangular digital x-ray sensor, as depicted in FIG. 1A. Alternatively, sensor bed 170 can be configured to hold a circular digital x-ray sensor. Rectangular and circular digital x-ray sensors are known in the art. Other shapes and sizes are also contemplated, as sensor bed 170 can be configured to receive a variety of types of digital x-ray sensors.

In one aspect, device 100 can accommodate a digital x-ray sensor having various shapes and sizes. Digital x-ray sensors available in sizes 0, 1, and 2 are contemplated for use with device 100. In some aspects, the digital x-ray sensor can be about 20 mm to about 50 mm in width and length, e.g., from about 20 mm to about 40 mm, or from about 20 mm to about 40 mm in width and/or length.

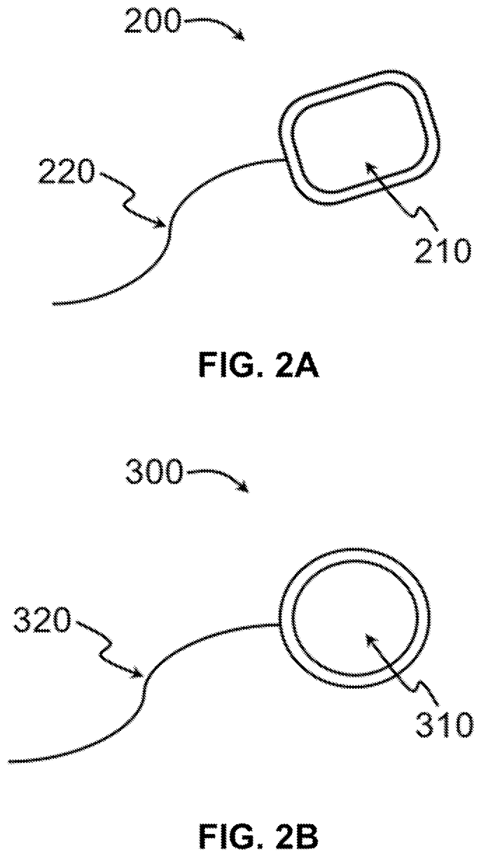

Referring to FIGS. 2A-B, a variety of digital x-ray sensors are contemplated for use with the intra-oral digital x-ray sensor device. As depicted in FIG. 2A, a generally rectangular digital x-ray sensor 200 can comprise a digital x-ray sensor body 210. Body 210 comprises internal circuitry and allows transmission of instructions and information to and from digital x-ray sensor 200 to an image rendering computer system. Cable 220 can be connected to digital x-ray sensor body 210 and allow for the transmission of instructions and information to and from the digital x-ray sensor. FIG. 2B depicts a circular digital x-ray sensor 300 comprising an x-ray sensor body 310 and a cable 320. As an alternative to cable 220, the digital x-ray sensor can be wireless and connect to an image rendering system such as a computer through bluetooth or another suitable wireless connection. In one aspect, x-ray sensor body 210 or x-ray sensor body 310 can be connected via cable 220 or 320 to a computing device, such as a desktop computer, laptop computer, or hand-held device or tablet. The sensor can in some aspects be connected to the computing device via a suitable connection, such as a USB connection. Thus, in some aspects, the x-ray sensor can be compatible with a variety of dental imaging software as one of skill in the art will appreciate. A non-limiting example of suitable dental imaging software is XV XRAY VISION 4, or APTERYX XRAYVISION, commercially avialable from Apteryx (Peoria, Ariz., U.S.A.).

In general, the intra-oral digital x-ray sensor device, which can comprise a built-in x-ray sensor, can help dental offices with time management, ensure adequate work flow, and provide a comfortable experience for the dental patient. The sensor can in some aspects be a high-contrast image such that high-contrast x-ray images can be generated from the intra-oral digital x-ray sensor device.

In some aspects, the intra-oral digital x-ray sensor device such as the one depicted FIGS. 1A-B can optionally be in digital communication with an intra-oral camera. A variety of intra-oral cameras are commercially available. In some aspects, by pairing or even physically connecting the intra-oral digital x-ray sensor device with an intra-oral camera, the dental technician can position the intra-oral digital x-ray sensor device in the appropriate location of the patient's mouth, depending on the nature of x-rays needed. Alternatively, as is typical with some conventional devices, the intra-oral digital x-ray sensor device described herein can be physically paired with a small ring that can be hooked onto the intra-oral digital x-ray sensor device and that can allow the technician to know where to take images while the intra-oral digital x-ray sensor device is in the patient's mouth. Thus, for example, with reference to FIGS. 1A-B, one or more openings or apertures (not shown) can be present within lingual inner side wall 110 and/or lingual outer side wall 120, to permit a small ring to be threaded through the sensor body such that the device can be positioned in an appropriate position in the patient's mouth.

The internal circuitry of digital x-ray sensors is known in the art. In one aspect, the digital x-ray sensor suitable for use with the intra-oral digital x-ray sensor device can comprise a series of components including without limitation a scintillator, a fiber optic lens array, a digital image sensor, and a circuit board. Some commercially available digital x-ray sensors also comprise a resilient material to absorb shock and protect internal components during movement and use. Scintillators of digital x-ray sensors can be reactive to x-ray emissions and are capable of producing visible light in proportion to the intensity of x-rays incident to the scintillator. Light produced by the scintillator can be directed through a fiber optic lens array and ultimately to an image sensor. The lensing array can include segments of optical fiber in parallel to the lensing array, which can direct light from the scintillator to the image sensor. Common image sensors include complementary metal-oxide-semiconductor (CMOS), which are capable of converting light intensity to a digital value corresponding to light intensity at certain pixel locations. Circuit boards of known digital x-ray sensors include control and power circuitry to operate the image sensor and data communication circuitry to transmit image data to an image rendering system such as a computer. Many digital x-ray sensors can be connected to an image rendering system from a cable through a suitable port, such as a USB port. Modern digital x-ray sensors typically include a USB cable attached to the sensor body, which can be connected to a suitable computer system.

A variety of commercially available digital x-ray sensors can be used with the disclosed intra-oral device. Non-limiting examples include ACTEON SOPIX2 Sensors, Dentsply Sirona Schick 33 Sensors, DEXIS Platinum Radiography Sensors, DEXIS Titanium Digital Sensors, Gendex GXS-700 Dental X-ray Sensors, KaVo IXS Digital Intraoral Sensors, Midmark CLEARVISION Digital Sensor Systems, and Planmecca PROSENSORs HD.

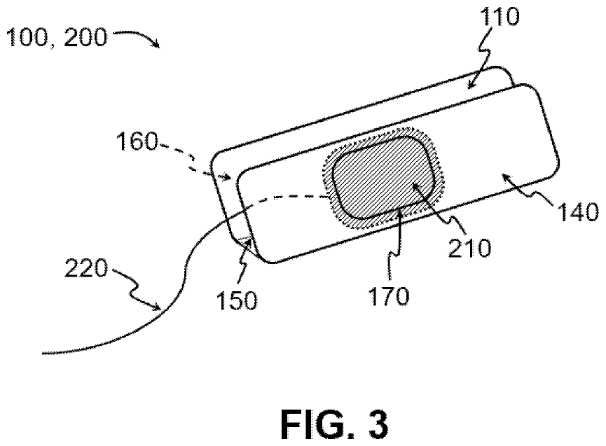

With reference to FIG. 3, body 210 of digital x-ray sensor 200 can be received within sensor bed 170 of digital x-ray sensor device 100. According to one aspect, body 210 of digital x-ray sensor 200 can conform to an internal cross-sectional area of sensor bed 170. Cable 220, which is connected to body 210 of digital x-ray sensor 200, can exit sensor bed 170 and connect to a suitable image rendering system such as a computer, e.g., through a USB connection (not shown). The digital x-ray sensor can be used according to methods known in the art, e.g., to render images of a patient's teeth or other dental features.

To provide comfort to the patient during use, the digital x-ray sensor device can be formed from a soft, flexible material such as a polymeric material, e.g., a thermoplastic elastomer. Suitable materials can be polymeric materials commonly used in dental applications. Non-limiting examples include ethylene vinyl acetate (EVA), polyolefin-based thermoplastic elastomers, polypropylene, polyvinylchloride, low density polyethylene, and high density polyethylene. The device can be made using methods known in the art, e.g., through molding or 3D-printing.

According to a further aspect, disclosed herein is a kit comprising the intra-oral digital x-ray sensor device together with one or more digital x-ray sensors. The digital x-ray sensors in the kit can be of various shapes and sizes to accommodate patients with different sized mouths. According to a further aspect, the kit can comprise instructions for using the digital x-ray sensor device and digital x-ray sensor to render a suitable image of a patient's teeth or dental work.

It will be apparent to those skilled in the art that various modifications and variations can be made without departing from the scope or spirit of this disclosure. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A dental x-ray sensor device comprising lingual and labial inner and outer side walls and a biting surface, the lingual and labial inner side walls and the biting surface together defining a channel configured to receive one or more teeth of a person; and a sensor bed between the labial inner and outer side walls configured to hold a digital x-ray sensor.

2. The device of claim 1, wherein the sensor bed is configured to hold a rectangular digital x-ray sensor.

3. The device of claim 1, wherein the sensor bed is configured to hold a circular digital x-ray sensor.

4. The device of claim 1, further comprising a digital x-ray sensor in the sensor bed, wherein the digital x-ray sensor conforms to an internal cross-sectional area of the bed.

5. The device of claim 4, wherein the digital x-ray sensor is rectangular.

6. The device of claim 4, wherein the digital x-ray sensor is circular.

7. The device of claim 4, further comprising a cable connected to the digital x-ray sensor, the cable being configured to transmit information to and from an image-rendering computer system.

8. The device of claim 1, wherein the channel is configured to receive one or more molar teeth of the person.

9. The device of claim 1, wherein the device is formed from a soft, flexible polymeric material.

10. The device of claim 1, wherein the device is formed from ethylene vinyl acetate (EVA).

11. A kit comprising:
a) a dental x-ray sensor device comprising lingual and labial inner and outer side walls and a biting surface, the lingual and labial inner side walls and the biting surface together defining a channel configured to receive one or more teeth of a person; and a sensor bed between the labial inner and outer side walls configured to hold a digital x-ray sensor; and
b) a digital x-ray sensor.

12. The kit of claim 11, wherein the sensor bed is configured to hold a rectangular digital x-ray sensor.

13. The kit of claim 11, wherein the sensor bed is configured to hold a circular digital x-ray sensor.

14. The kit of claim 11, wherein the channel is configured to receive one or more molar teeth of the person.

15. The kit of claim 11, wherein the device is formed from a soft, flexible polymeric material.

16. The kit of claim 11, wherein the device is formed from ethylene vinyl acetate (EVA).
